(19) United States
(12) Patent Application Publication
(10) Pub. No.: US 2016/0287565 A1
(43) Pub. Date: Oct. 6, 2016

(54) COMBINATION THERAPY FOR TREATING DISORDERS ASSOCIATED WITH EXCESS CORTISOL PRODUCTION

(71) Applicant: Millendo Therapeutics, Inc., Ann Arbor, MI (US)

(72) Inventors: Pharis Mohideen, Pine Brook, NJ (US); Julia Christine Owens, Ann Arbor, MI (US)

(21) Appl. No.: 15/092,266

(22) Filed: Apr. 6, 2016

Related U.S. Application Data
(60) Provisional application No. 62/143,713, filed on Apr. 6, 2015.

(51) Int. Cl. A61K 31/444; A61K 31/4188; A61K 45/06; A61K 31/17

(52) U.S. Cl. CPC A61K 31/444; A61K 31/17; A61K 31/4188; A61K 45/06

(57) ABSTRACT

Methods are provided for treatment of disorders associated with excess cortisol production, including, but not limited to, treatment of Cushing's syndrome. Such methods involve administration of a therapeutically effective amount of a combination of: (a) an inhibitor of CYP11B1; and (b) ACAT1 inhibitor N-(2,6-bis(1-methylethyl)phenyl)-N′-((1-(4-(dimethyl-lamino)phenyl)cyclopentyl)-methyl)urea or a salt thereof; or (a) an inhibitor of CYP11B1; (b) an inhibitor of CYP11B2; and (c) ACAT1 inhibitor N-(2,6-bis(1-methylethyl)phenyl)-N′-((1-(4-(dimethyl-lamino)phenyl)cyclopentyl)-methyl)urea or a salt thereof.

### CROSS REFERENCE TO RELATED APPLICATION

This application claims the benefit under 35 U.S.C. §119(e) to U.S. Provisional Application No. 62/143,713 filed on Apr. 6, 2015, which is incorporated by reference herein it its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120205_407_SEQUENCE_LISTING.txt. The text file is 25.2 KB, was created on Apr. 4, 2016, and is being submitted electronically via EFS-Web.

### BACKGROUND

1. Technical Field

Methods and agents are provided for treatment of disorders associated with excess cortisol production, while ameliorating the adverse effects associated with increased androgen and mineralocorticoid activity induced by such treatment.

2. Description of the Related Art

Hypercortisolism refers to a range of conditions characterized by excess production of cortisol in the body. Cushing's syndrome, arises due to prolonged exposure to excess cortisol. Exogenous Cushing's syndrome is caused by treatment with exogenous glucocorticoids. Endogenous Cushing's syndrome results from dysfunction of the body's own system of secreting cortisol. Endogenous Cushing's syndrome is classified as either ACTH dependent or ACTH independent.

ACTH independent Cushing's syndrome is usually due to a primary adrenocortical neoplasm, either an adenoma or carcinoma, characterized by chronic cortisol hypersecretion. ACTH-secreting neoplasms cause ACTH dependent Cushing's syndrome. ACTH hypersecretion stimulates the growth of the adrenal glands and the hypersecretion of corticosteroids. An anterior pituitary tumor is the most common cause of ACTH dependent Cushing's syndrome, and is known as Cushing's disease. Non-pituitary ectopic sources of ACTH include thymoma, medullary carcinoma of the thyroid, pheochromocytoma, islet cell tumors of the pancreas, oat cell carcinoma, small-cell lung carcinoma, and carcinoid tumor.

Symptoms of Cushing's syndrome include, but are not limited to: rapid weight gain, particularly in the trunk and face (central obesity); growth of fat pads on the collarbone, on the back of the neck (“buffalo hump”), and on the face (“moon face”); excess sweating; dilation of capillaries; thinning of the skin (causing easy bruising and dryness); purple or red striae on the trunk, buttocks, arms, legs, or breasts; proximal muscle weakness, hirsutism; baldness; insomnia; impotence; amenorrhoea/oligomenorrhea; infertility; memory and attention dysfunction; depression; anxiety; acne; persistent hypertension; hypercholesterolemia; insulin resistance; polyuria; diabetes mellitus; and osteoporosis. Untreated Cushing's syndrome can lead to heart disease and increased mortality.

Current drugs used for treatment of Cushing's syndrome include agents that inhibit 11-beta-hydroxylase activity, the final step in cortisol synthesis. 11-beta-hydroxylase inhibitors include for example, metyrapone and etomidate. However, 11-beta-hydroxylase inhibitors have troublesome adverse effects resulting from increases in androgen and mineralocorticoid precursors. Inhibition of 11-beta-hydroxylase may result in build-up of 11-deoxysteroids before the enzyme blockade or shunting of 11-deoxysteroid precursors to the androgen or mineralocortiocoid synthetic pathways, which are proximal to the blockade. Hypertension, edema, acne, hirsutism, hypokalemia, and other adverse effects may occur from hyperandrogenism or hypermineralocorticism.

While advances have been made in this field, there remains a need in the art for additional methods and agents for treatment of disorders associated with excess cortisol production, including but not limited to Cushing's syndrome, while ameliorating the adverse effects associated with some currently used treatments. The present invention fulfills these needs and provides further related advantages.

### BRIEF SUMMARY

In brief, methods and combination of agents are provided for treatment of disorders associated with excess cortisol production, including (but not limited to) Cushing's syndrome, while ameliorating the adverse effects associated with increased androgen and mineralocorticoid activity induced by such treatment. A CYP11B1 inhibitor reduces cortisol production by inhibiting 11-beta-hydroxylase activity. However, such blockade of 11-beta-hydroxylase result in build-up of 11-deoxysteroids (11-deoxycortisol, 11-deoxycorticosterone, or both) shunting of 11-deoxysteroid precursors to the androgen synthetic pathway, mineralocorticoid synthetic pathway, or both. The present disclosure provides the combination of a CYP11B1 inhibitor with an ACAT1 inhibitor, and optionally a CYP11B2 inhibitor. The ACAT1 inhibitor acts upstream of adrenal steroid biosynthetic pathways, inhibiting the esterification of free cholesterol into cholesteryl esters. Cholesterol esters are stored as cytoplasmic lipid droplets in the cell. In steroidogenic tissues such as the adrenal gland, cholesterol esters act as a cholesterol reservoir for biosynthesis of steroid hormones. Enzymatic conversion of cholesterol to pregnenalone by CYP11A1 is the rate limiting step for steroid biosynthesis. Reduction of cholesterol esters by the ACAT1 inhibitor provides for reducing production of androgen and mineralocorticoid precursors that result from 11-beta hydroxylase blockade.

In one aspect, the present disclosure provides a method for treatment a disorder associated with excess cortisol production in a subject in need thereof, comprising administering to a subject a therapeutically effective amount of a combination of: (a) a CYP11B1 inhibitor; and (b) an ACAT1 inhibitor, wherein the ACAT1 inhibitor is N-(2,6-bis(1-methylethyl)phenyl)-N′-((1-(4-(dimethylamino)phenyl)cyclopentyl)-methyl)urea or a salt thereof.

In certain embodiments, the method further comprises administering a CYP11B2 inhibitor. In some embodiments, the CYP11B1 inhibitor and CYP11B2 inhibitor are in the form of a dual CYP11B1/CYP11B2 inhibitor.

In certain embodiments, the dual CYP11B1/CYP11B2 inhibitor is osilodrostat.

In certain embodiments, the CYP11B1 inhibitor is metyrapone.

In certain embodiments, the CYP11B1 inhibitor is not an adrenolytic agent, for example, mitotane.

In certain embodiments, the disorder associated with excess cortisol production is Cushing's syndrome.

In certain embodiments, the administration of the combination of CYP11B1 inhibitor and ACAT1 inhibitor N-(2,6-bis(1-methylethyl)phenyl)-N′-((1-(4-(dimethyl-amino)phenyl)cyclopentyl)-methyl)urea or a salt thereof decreases the production or activity of at least one androgen or precursor thereof, mineralocorticoid or a precursor thereof, glucocorticoid precursor, or any combination thereof as compared to administration of the CYP11B1 inhibitor alone. In other embodiments, administration of CYP11B1 inhibitor, CYP11B2 inhibitor, and ACAT1 inhibitor N-(2,6-bis(1-methylethyl)phenyl)-N′-((1-(4-(dimethyl-amino)phenyl)cyclopentyl)-methyl)urea or a salt thereof decreases the production or activity of at least one androgen or precursor thereof, mineralocorticoid or a precursor thereof, glucocorticoid precursor, or any combination thereof as compared to administration of the CYP11B1 inhibitor and CYP11B2 inhibitor alone. In certain embodiments, the androgen or precursor thereof is testosterone, androstenedione, DHEA, DHEA-S, or a combination thereof. In certain embodiments, the mineralocorticoid or precursor thereof is corticosterone, 11-deoxycorticosterone, aldosterone, or any combination thereof. In certain embodiments, the glucocorticoid precursor is 11-deoxycortisol.

In certain embodiments, the administration of CYP11B1 inhibitor and ACAT1 inhibitor N-(2,6-bis(1-methylethyl)phenyl)-N′-((1-(4-(dimethylamino)phenyl)-cyclopentyl)-methyl)urea or a salt thereof decreases an adverse effect associated with administration of CYP11B1 inhibitor alone. In other embodiments, the administration of CYP11B1 inhibitor, CYP11B2 inhibitor, and ACAT1 inhibitor N-(2,6-bis(1-methylethyl)phenyl)-N′-((1-(4-(dimethylamino)phenyl)-cyclopentyl)-methyl)urea or a salt thereof decreases an adverse effect associated with administration of CYP11B1 inhibitor and CYP11B2 inhibitor alone. In certain embodiments, the adverse effect is acne, hirsutism, virilization, menstrual irregularity, infertility due to anovulation, enlarged clitoris, male infertility, hypertension, edema, hypokalemia, or any combination thereof.

In certain embodiments, the administration of CYP11B1 inhibitor and ACAT1 inhibitor N-(2,6-bis(1-methylethyl)phenyl)-N′-((1-(4-(dimethylamino)phenyl)-cyclopentyl)-methyl)urea or a salt thereof reduces cholesterol ester levels in adrenocortical cells as compared to adrenocortical cells treated with CYP11B1 inhibitor alone. In other embodiments, the administration of CYP11B1 inhibitor, CYP11B2 inhibitor, and ACAT1 inhibitor N-(2,6-bis(1-methylethyl)phenyl)-N′-((1-(4-(dimethylamino)phenyl)-cyclopentyl)-methyl)urea or a salt thereof reduces cholesterol ester levels in adrenocortical cells as compared to adrenocortical cells treated with CYP11B1 inhibitor and CYP11B2 inhibitor alone.

In certain embodiments, the administration of CYP11B1 inhibitor and ACAT1 inhibitor N-(2,6-bis(1-methylethyl)phenyl)-N′-((1-(4-(dimethylamino)phenyl)-cyclopentyl)-methyl)urea or a salt thereof increases reduction of cortisol biosynthesis as compared to administration of the CYP11B1 inhibitor alone. In other embodiments, the administration of CYP11B1 inhibitor, CYP11B2 inhibitor, and ACAT1 inhibitor N-(2,6-bis(1-methylethyl)phenyl)-N′-((1-(4-(dimethylamino)phenyl)-cyclopentyl)-methyl)urea or a salt thereof increases reduction of cortisol biosynthesis as compared to administration of the CYP11B1 inhibitor and CYP11B2 inhibitor alone.

In certain embodiments, the CYP11B1 inhibitor and ACAT1 inhibitor are administered simultaneously or sequentially. In certain embodiments, the CYP11B1 inhibitor and ACAT1 inhibitor are administered in separate formulations. In other embodiment, the CYP11B1 inhibitor and ACAT1 inhibitor are administered simultaneously in the same formulation.

In certain embodiments, the CYP11B1 inhibitor, CYP11B2 inhibitor, and ACAT1 inhibitor are each administered simultaneously or sequentially. In certain embodiments, the CYP11B1 inhibitor, CYP11B2 inhibitor, and ACAT1 inhibitor are each administered in separate formulations. In other embodiments, the CYP11B1 inhibitor and CYP11B2 inhibitor are administered in the same formulation and the ACAT1 inhibitor is administered in a separate formulation. In other embodiments, the CYP11B1 inhibitor, CYP11B2 inhibitor, and ACAT1 inhibitor are each administered simultaneously in the same formulation.

Additional aspects of the present disclosure provide for pharmaceutical compositions comprising a therapeutically effective amount of a combination of a CYP11B1 inhibitor and ACAT1 inhibitor N-(2,6-bis(1-methylethyl)phenyl)-N′-((1-(4-(dimethylamino)phenyl)cyclopentyl)-methyl)urea or a salt thereof, optionally further comprising a CYP11B2 inhibitor, for treating a disorder associated with excess cortisol production, and kits with unit doses of the combination of agents described herein, usually in oral or injectable doses, for use in treating a disorder associated with excess cortisol production in a subject in need thereof.

These and other aspects of the invention will be evident up references to the attached figures and following detailed description.

### BRIEF DESCRIPTION OF THE SEVERAL VIEWS OF THE DRAWINGS

FIG. 1 shows steroid biosynthetic pathways in the adrenal gland, with the major classes of steroid hormones, individual steroids and intermediates, and enzymatic pathways.

FIGS. 2A-D are bar graphs depicting hormone, precursors, and renin levels in human, adult patients with Cushing's disease treated with maximal dose of 100 mg/day (b.i.d.) of osilodrostat (LCI699) for 70 days (n=12). Levels of (A) ACTH, (B) 11-deoxycortisol, (C) testosterone (in 8 females), and (D) 11-deoxycorticosterone (“DOC”) levels were measured at days 1, 70, and 84 of treatment. ULN=upper limit of normal. All data are mean±SEM.

FIG. 3 is a line graph showing individual changes in testosterone levels in human, adult patients (5 males and 12 females) with Cushing's disease who completed a 22 week treatment course with osilodrostat (LCI699). Each line represents an individual patient. Normal ranges are as follows: males, 8.7-38.2 nmol/L; females, 0.1-1.6 nmol/L

FIG. 4 shows effects of ATR-101 treatment in dogs on production of steroids and their intermediates. Changes in steroid and steroid intermediate levels are shown as % reduction as compared to maximum level as measured on Day 0. *Day 1 data used for maximum steroid level. **Day 3 data used for maximum steroid level.

FIGS. 5A-B show illustrative transcript and polypeptide sequences for human CYP11B1 (SEQ ID NOS: 1 and 2, respectively).

FIGS. 6A-B show illustrative transcript and polypeptide sequences for human ACAT1 (SEQ ID NOs: 3 and 4, respectively).

### DETAILED DESCRIPTION

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

As mentioned above, methods and agents are provided for treatment of disorders associated with excess cortisol production. Such methods involve administering to a subject in need of such treatment a therapeutically effective amount of a combination of agents as defined in more detail below.

As used herein, “treatment” includes therapeutic applications to slow or stop progression of a disorder associated with excess cortisol production, prophylactic application to prevent development of a disorder associated with excess cortisol production, and reversal of a disorder associated with excess cortisol production. Reversal of a disorder differs from a therapeutic application which slows or stops a disorder in that with a method of reversing, not only is progression of a disorder completely stopped, cellular behavior is moved to some degree, toward a normal state that would be observed in the absence of excess cortisol production.

As used herein, “Cushing's syndrome” means a hormonal disorder caused by prolonged exposure of the body's tissues to high levels of cortisol. Cushing's syndrome is sometimes referred to as “hypercortisolism” (excess cortisol production). Cushing's syndrome includes various subtypes of the disease, including Cushing's disease, adrenal Cushing's syndrome, and ectopic ACTH syndrome, which are categorized by the cause of hypercortisolism. Cushing's disease, also known as pituitary Cushing's, is caused by a pituitary gland tumor which secretes excessive ACTH, which in turn stimulates the adrenal glands to make more cortisol. Ectopic ACTH syndrome is caused by tumors that arise outside the pituitary gland that can produce ACTH, which stimulates cortisol production. Adrenal Cushing's syndrome is caused by an abnormality of the adrenal gland, usually an adrenal tumor, which causes excess cortisol secretion.

As used herein, “subclinical hypercortisolism,” also known as “preclinical” or “subclinical Cushing's syndrome,” refers to a condition of biochemical cortisol excess without the classical signs or symptoms of overt hypercortisolism (e.g., purple striae, easy bruising, proximal muscle weakness) (reviewed by Chiodini et al., (2011) J. Clin. Endocrinol. Metab. 96:1223-1236).

As used herein, a “subject in need thereof” refers to a subject at risk of, or suffering from, a disease, disorder or condition (e.g., Cushing's syndrome) that is amenable to treatment or amelioration with the combination of agents thereof provided herein. In certain embodiments, a subject in need is a mammal. A “mammal” includes humans and both domestic animals, such as laboratory animals and household pets (e.g., cats, dogs, swine, cattle, sheep, goats, horses, rabbits), and non-domestic animals, such as wildlife or the like. In certain embodiments, a human subject may be a child, an adolescent (i.e., generally a subject who is at least 12 years old), or an adult. In certain embodiments, a human subject may be female or male.

As used herein, the phrase term “therapeutically effective amount” refers to an amount of a therapeutic agent to treat, ameliorate, or prevent a disease or condition, or to exhibit a detectable therapeutic or preventative effect. The effect is detected by, for example, a reduction in cortisol production. The effect is also detected by, for example, steroid levels or steroid intermediate levels. Therapeutic effects also include reduction in physical symptoms, such as hypertension, impaired glucose tolerance, hyperlipidemia, etc. The precise effective amount for a subject will depend upon the subject's size and health, the nature and extent of the condition, the therapeutics or combination of therapeutics selected for administration, and other variables known to those of skill in the art. The effective amount for a given situation is determined by routine experimentation and is within the judgment of the clinician. In reference to a combination, a therapeutically effective dose refers to combined amounts of the active ingredients that result in the therapeutic effect, whether administered serially, concurrently or simultaneously.

An “agent” means a compound that exhibits the characteristics (e.g., inhibition of 11β-hydroxylase activity) disclosed herein. The agent itself can be the active form, or the agent can be metabolized upon administration to the subject to yield the active form. Thus, as used herein, the term agent also includes a prodrug. To this end, a “prodrug” is a compound typically having little or no pharmacological activity itself but capable of releasing, for example by hydrolysis or metabolic cleaving of a linkage such as an ester moiety, an active agent upon administration to the subject.

As used herein, “CYP11B1 inhibitor” means an agent that inhibits or reduces human steroid 11β-hydroxylase activity encoded by the CYP11B1 gene. CYP11B1 encodes steroid 11β-hydroxylase, also known as cytochrome P450 family 11, subfamily B, polypeptide 1, which is a steroid hydroxylase found in the zona glomerulosa and zona fasciculata of the adrenal gland. 11β-hydroxylase converts 11-deoxycortisol to cortisol. An exemplary nucleotide sequence for CYP11B1 is provided by Genbank Accession NM_000497 (SEQ ID NO:1). An exemplary amino acid sequence for CYP11B1 is provided by Genbank Accession NP_000488 (SEQ ID NO:2). In certain embodiments, a CYP11B1 inhibitor exhibits an IC50 value against CYP11B1 of less than 1 μM. In certain embodiments, a CYP11B1 inhibitor may be a selective inhibitor of 11β-hydroxylase activity encoded by the CYP11B1 gene. In other embodiments, a CYP11B1 inhibitor may also inhibit or reduce 11β-hydroxylase activity encoded by the CYP11B2 gene, which converts 11-deoxycorticosterone to corticosterone (a “dual CYP11B1/CYP11B2 inhibitor”).

As used herein, a “CYP11B2 inhibitor” means an agent that inhibits or reduces steroid 11β-hydroxylase or 18-β-hydroxylase activity encoded by the CYP11B2 gene. CYP11B2 encodes 11/18-beta-hydroxylase, also known as cytochrome P450 family 11 subfamily B, polypeptide 2 or aldosterone synthase, which is an enzyme found in the zona glomerulosa of the adrenal cortex. In certain embodiments, a CYP11B2 inhibitor inhibits both 11-β-hydroxylase and 18-β-hydroxylase activity encoded by the CYP11B2. In other embodiments, a CYP11B2 inhibitor inhibits 11-β-hydroxylase encoded by the CYP11B2 gene. In certain embodiments, a CYP11B2 inhibitor exhibits an IC50 value against CYP11B2 of less than 1 μM. In certain embodiments, a CYP11B2 inhibitor may be a selective inhibitor of 11β-hydroxylase activity encoded by the CYP11B2 gene. In other embodiments, a CYP11B2 inhibitor may also inhibit or reduce 11-β-hydroxylase activity encoded by the CYP11B1 gene (a “dual CYP11B1/CYP11B2 inhibitor”).

As used herein, an “ACAT1 inhibitor” means an agent that inhibits or reduces human acyl coenzyme A:cholesterol acyltransferase1 (huACAT1) activity. ACAT1, also known as sterol o-acyltransferase1 (SOAT1), catalyzes the esterification of free cholesterol into cholesteryl esters. An exemplary nucleotide sequence for ACAT1 is provided by Genbank Accession # L21934.2 (SEQ ID NO:3). An exemplary amino acid sequence for ACAT1 is provided by Genbank Accession # AAC37532.2 (SEQ ID NO:4). In certain embodiments, an ACAT1 inhibitor exhibits an IC50 value against huACAT1 of less than 10 μM determined by the fluorescent cell-based assay measuring esterification of NBD-cholesterol in AC29 cells expressing huACAT1 as described in Lada et al. (J. Lipid Res. 45:378-386, 2004) (incorporated by reference herein in its entirety).

In certain embodiments, an ACAT1 inhibitor is N-(2,6-bis(1-methylethyl)-phenyl)-N′-((1-(4-(dimethylamino)phenyl)cyclopentyl)-methyl)urea or a salt thereof. A monohydrochloride salt of the free base, referred to herein as “ATR-101” is depicted by the following structure:

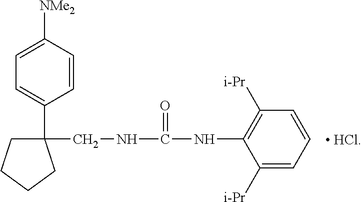

As used herein, the term “derivative” refers to a modification of a compound by chemical or biological means, with or without an enzyme, which modified compound is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.” A derivative may have different chemical, biological or physical properties from the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiquitination, esterification, and amidation.

The present disclosure provides methods for treating a disorder associated with excess cortisol production, including but not limited to Cushing's syndrome, excess cortisol production, subclinical hypercortisolism, and symptoms associated with excess cortisol production in a subject. Current agents that inhibit steroidogenesis include those that inhibit 11-beta-hydroxylase activity, such as metyrapone, etomidate, and trilostane. Blockade of 11-beta-hydroxylase results in build-up of 11-deoxysteroids (11-deoxycortisol, 11-deoxycorticosterone, or both) and may result in shunting of 11-deoxysteroid precursors to the androgen synthetic pathway, mineralocorticoid synthetic pathway, or both.

In order to ameliorate the adverse effects associated with increased androgen and mineralocorticoid activity induced by 11-beta hydroxylase inhibition, the present disclosure proposes to combine a CYP11B1 inhibitor with an ACAT1 inhibitor, and optionally a CYP11B2 inhibitor, wherein the ACAT1 inhibitor is N-(2,6-bis(1-methylethyl)phenyl)-N′-((1-(4-(dimethylamino)phenyl)cyclopentyl)-methyl)urea or a salt thereof, for administration to a subject. An ACAT1 inhibitor, e.g., N-(2,6-bis(1-methylethyl)phenyl)-N′-((1-(4-(dimethylamino)phenyl)cyclopentyl)-methyl)urea or a salt thereof, acts upstream of adrenal steroid biosynthetic pathways, inhibiting the esterification of free cholesterol into cholesteryl esters. Cholesterol esters are stored as cytoplasmic lipid droplets in the cell. In steroidogenic tissues such as the adrenal gland, cholesterol esters act as a cholesterol reservoir for biosynthesis of steroid hormones. Enzymatic conversion of cholesterol to pregnenalone by CYP11A1 is the rate limiting step for steroid biosynthesis. Reduction of cholesterol esters by N-(2,6-bis(1-methylethyl)phenyl)-N′-((1-(4-(dimethylamino)phenyl)-cyclopentyl)-methyl)urea or a salt thereof provides a method for reducing production of androgen and mineralocorticoid precursors that result from 11-beta hydroxylase blockade.

In one aspect, the present disclosure provides a method for treating a disorder associated with excess cortisol production in a subject in need thereof, comprising administering to a subject a combination of therapeutically effective amounts of a CYP11B1 inhibitor, and an ACAT1 inhibitor, wherein the ACAT1 inhibitor is N-(2,6-bis-(1-methylethyl)phenyl)-N′-((1-(4-(dimethylamino)phenyl)cyclopentyl)-methyl)urea or a salt thereof. In certain embodiments, the method further comprises administering a CYP11B2 inhibitor. In some embodiments, the CYP11B1 inhibitor and CYP11B2 inhibitor are in the form of a dual CYP11B1/CYP11B2 inhibitor.

CYP11B1 gene encodes steroid 11 beta-hydroxylase. Steroid 11 beta-hydroxylase (P-450(11) beta) is a mitochondrial cytochrome P-450 enzyme expressed in the zona fasciculata and zone reticularis of the adrenal cortex necessary for cortisol biosynthesis, converting 11-deoxycortisol to cortisol. An exemplary nucleic acid sequence for CYP11B1 is provided by Genbank Accession No. NM_000497 (SEQ ID NO:1). An exemplary amino acid sequence is provided by Genbank Accession No. NP_000488 (SEQ ID NO:2). Transcript variants encoding different isoforms have been described for CYP11B1.

CYP11B2, which encodes aldosterone synthase (also known as steroid 11/18-β-hydroxylase), is closely related to CYP11B1. Aldosterone synthase is normally expressed in the zona glomerulosa and catalyzes three reactions for the production of the mineralocorticoid aldosterone: the 11-beta-hydroxylation of 11-deoxycorticosterone (11-DOC) to corticosterone; the 18-hydroxylation of corticosterone to 18-hydroxycorticosterone (18-OHB); and the 18-oxidation of 18-hydroxycorticosterone to aldosterone. An exemplary nucleic acid sequence for CYP11B2 is provided by Genbank Accession No. NM_000498.3 (SEQ ID NO:5). An exemplary amino acid sequence for CYP11B2 is provided by Genbank Accession No. NP_000489.3 (SEQ ID NO:6).

Transcript variants encoding different isoforms have been described for CYP11B1. The encoded proteins of CYP11B1 and CYP11B2 show 93% identity and are encoded on the same chromosome. The difference in expression pattern in the adrenal cortex is due to the regulatory regions of the two genes. The promoter region of CYP11B2 is regulated by angiotensin II and potassium, while the promoter region of CYP11B1 is responsive to ACTH. Due to the high sequence identity, identification of selective inhibitors of one enzyme versus the other is particularly challenging. Recently, however, selective inhibition of CYP11B1 has been demonstrated. Methods for determining inhibition of CYP11B1 or CYP11B2 are known in the art (see, e.g., Ehmer et al., (2002) J. Steroid Biochem. Mol. Biol. 81:173-179). In certain embodiments, a CYP11B1 inhibitor may also inhibit or reduce 11/18-beta-hydroxylase activity encoded by the CYP11B2 gene. In other embodiments, a CYP11B1 inhibitor is a selective CYP11B1 inhibitor.

A CYP11B1 inhibitor is an agent that inhibits or reduces human steroid 11β-hydroxylase activity encoded by the CYP11B1 gene. 11β-hydroxylase converts 11-deoxycortisol to cortisol. CYP11B1 inhibitors that may be used in the methods described herein include, for example: metyrapone or a derivative thereof, osilodrostat (also known as LCI699) or a derivative thereof (see, U.S. Pat. No. 8,609,862; see also, Bertagna et al., J. Clin. Endocrinol. Metab. (2014) 99:1375-1383), etomidate or a derivative thereof, etomidate derivative compound 33 described in Hille et al. (2011) ACS Med. Chem. Lett. 2:2-6; compound 23 (2-(1H-imidazol-1-yl)-1-(4-{[3(trifluoromethoxy)benzyl]oxy}phenyl) ethanone; see, Stefanachi et al., Eur. J. Med. Chem. (2015) 89:106-14); FAD286 (see, LaSala et al. (2009) Anal. Biochem. 394:56-61); triazole compounds described in Hoyt et al., 2015, ACS Med. Chem. Lett. 6:861-5; derivatives of etomidate described in Zolle et al., J. Med. Chem. (2008) 51:2244-2253); imidazole compounds described in PCT Publication WO2012/052540; aromatic compounds described in U.S. Patent Publication 2009/0105278; and imidazole derivatives described in U.S. Pat. No. 8,436,035 (each reference incorporated by reference herein in its entirety). In certain embodiments, the CYP11B1 inhibitor is osilodrostat (LCI699). In other embodiments, the CYP11B1 inhibitor is metyrapone.

A CYP11B2 inhibitor is an agent that inhibits or reduces human steroid 11-β-hydroxylase activity, 18-β-hydroxylase activity, or both encoded by the CYP11B2 gene. CYP11B2 inhibitors that may be used in the methods described herein, include for example, those compounds described in: Hartmann et al., 2003, Eur. J. Med. Chem. 38:363-6; Hoyt et al., 2015, ACS Med. Chem. Lett. 6:861-865; Martin et al., 2015, J. Med. Chem. 58:8054-65; Hoyt et al., 2015, ACS Med. Chem. Lett. 6:573-8; and U.S. Pat. No. 8,541,404 (each reference incorporated by reference herein in its entirety). A CYP11B2 inhibitor may be administered as a separate agent from the CYP11B1 inhibitor or may be in the form of a dual CYP11B1/CYP11B2 inhibitor.

A dual CYP11B1/CYP11B2 inhibitor is an agent that inhibits or reduces human 11-β-hydroxylase activity encoded by the CYP11B1 gene and CYP11B2 gene. A dual CYP11B1/CYP11B2 inhibitor may have stronger inhibitory activity towards CYP11B1 than CYP11B2, stronger inhibitory activity towards CYP11B2 than CYP11B1, or equivalent inhibitory activity towards both CYP11B1 and CYP11B2. Dual CYP11B1/CYP11B2 inhibitors that may be used in the methods described herein include for example, osilodrostat (also known as LCI699) or a derivative thereof (U.S. Pat. No. 8,609,862; Bertagna et al., J. Clin. Endocrinol. Metab. (2014) 99:1375-1383) and those compounds described in Meredith et al., 2013 (ACS Med. Chem. Lett. 4:1203-1207) and U.S. Patent Publication 2016/0002207 (each reference incorporated by reference herein in its entirety). In certain embodiments, a dual CYP11B1/CYP11B2 inhibitor is osilodrostat (LCI699).

Metyrapone is depicted by the following structure:

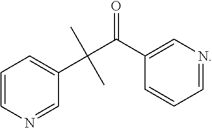 [SMILES: CC(C)(C(=O)c1cccnc1)c1cccnc1]

Osilodrostat (LCI699) is depicted by the following structure:

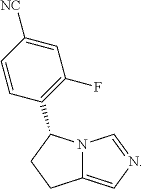 [SMILES: N#Cc1ccc([C@H]2CCc3cncn32)c(F)c1]

In certain embodiments, reference to a CYP11B1 inhibitor does not include an adrenolytic agent, for example, mitotane.

Acyl-coenzyme A:cholesterol transferase (ACAT) is an integral membrane protein localized in the endoplasmic reticulum. ACAT catalyzes formation of cholesteryl esters (CE) (also known as cholesterol esters) from cholesterol and fatty acyl coenzyme A. Cholesteryl esters are stored as cytoplasmic lipid droplets in the cell. In steroidogenic tissues, such as the adrenal gland, cholesteryl esters act as a cholesterol reservoir for biosynthesis of steroid hormones. In mammals, there are two ACAT isoenzymes, ACAT1 and ACAT2. ACAT2 is expressed in the liver and intestine. In humans, ACAT1 expression is most highly expressed in adrenal glands over other tissues. ACAT1 is the main isoenzyme in the adrenal gland. The major isoform of ACAT1 is a 50 kDa protein. ACAT1 may also be present as a minor 56 kDa protein.

N-(2,6-bis(1-methylethyl)phenyl)-N′-((1-(4-(dimethylamino)phenyl)-cyclopentyl)-methyl)urea or a salt thereof has been previous described (see, e.g., Trivedi, B. K., et al., (2004) J. Med. Chem., 37:1652-1659; U.S. Pat. No. 5,015,644). The monohydrochloride salt (as depicted above) is referred to herein as “ATR-101.” In addition to the monohydrochloride salt, other contemplated salt forms include salts which retain biological effectiveness and which are not biologically or otherwise undesirable, and which are formed with inorganic acids such as, but not limited to, hydrochloric acid, hydrobromic acid, sulfuric acid, nitric acid, phosphoric acid and the like, or with organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

In the methods described herein, an ACAT inhibitor inhibits the ability of human ACAT1 to catalyze the esterification of free cholesterol into cholesteryl ester. An agent's inhibitory activity and IC50 may be measured using methods known in the art, for example a fluorescent cell-based assay measuring esterification of NBD-cholesterol in AC29 cells expressing huACAT1 as described in Lada et al. (J. Lipid Res. 45:378-386, 2004) (incorporated by reference herein in its entirety). AC29 cells lack endogenous ACAT1 activity and are transfected to express human ACAT1. The assay uses 22-[N-(7-nitrobenz-2-oxa-1,3-diazol-4-yl)amino]-23,24-bisnor-5-cholen-3-ol (NBD-cholesterol), a fluorescent sterol analog in which the NBD moiety replaces the terminal segment of the alkyl tail of cholesterol. NBD-cholesterol has been shown to mimic native cholesterol absorption in multiple systems. In a polar environment, NBD-cholesterol is weakly fluorescent. In a nonpolar environment, NBD-cholesterol is strongly fluorescent. The fluorescent property of NBD-cholesterol is used to measure ACAT activity, as cholesterol is a polar lipid and cholesteryl ester is nonpolar. Untransfected AC29 cells or AC29 cells expressing huACAT1 treated with a known ACAT inhibitor can be used to determine background fluorescence due to free-NBD-cholesterol.

A disorder associated with excess cortisol production that may be treated using the methods described herein include, for example: Cushing's syndrome (ACTH dependent (e.g., Cushing's disease) or ACTH independent); excess cortisol production, subclinical hypercortisolism, and symptoms associated with excess cortisol production. In certain embodiments, the disorder associated with excess cortisol production is Cushing's syndrome.

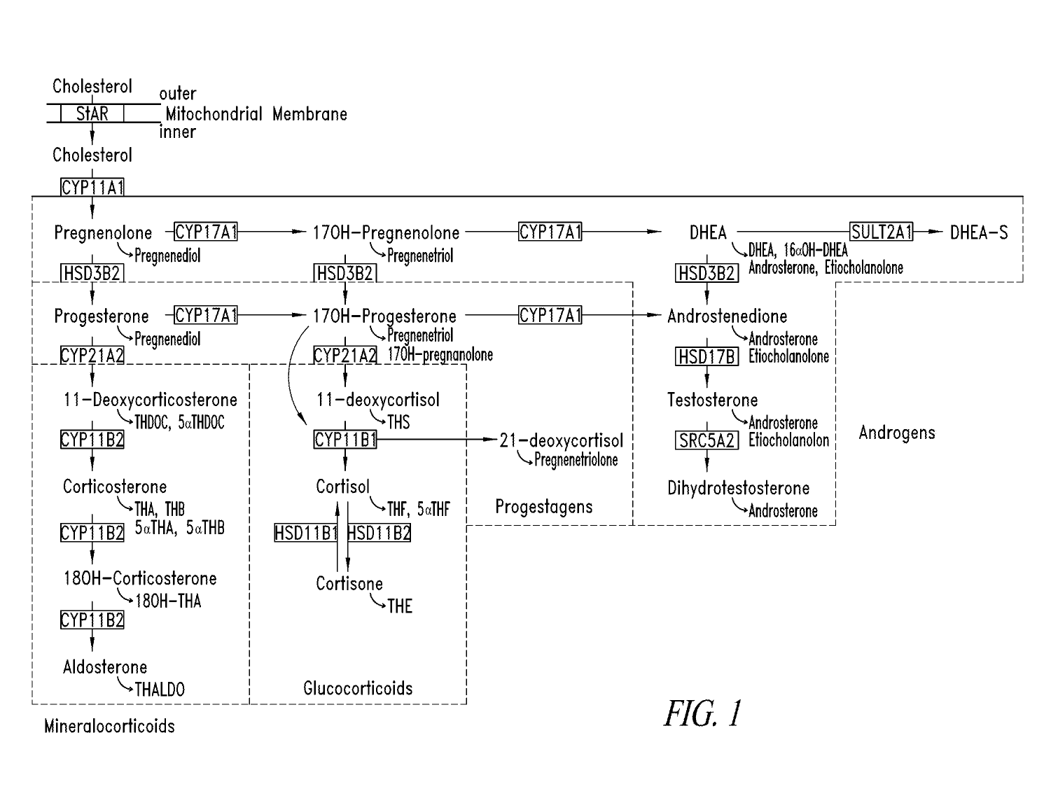

In ACTH-dependent disease, inhibition of cortisol production by a CYP11B1 inhibitor and resulting decrease in serum cortisol results in increased secretion of ACTH. Increased ACTH may cause overproduction of steroid precursors (before the 11-beta hydroxylase block). These steroid precursors may be shunted into mineralocorticoid or androgen synthesis pathways, which are proximal to the 11-beta hydroxylase blockade (see, FIG. 1, which shows adrenal steroid biosynthetic pathways).

In certain embodiments, the administration of the combination of a CYP11B1 inhibitor and ACAT1 inhibitor N-(2,6-bis(1-methylethyl)phenyl)-N′-((1-(4-(dimethylamino)phenyl)cyclopentyl)-methyl)urea or a salt thereof decreases the production of at least one androgen or a precursor thereof, mineralocorticoid or a precursor thereof, 11-deoxycortisol, or any combination thereof, as compared to administration with the CYP11B1 inhibitor alone. Methods of measuring steroid hormones or their precursors are known in the art, and primarily use blood, urine, or saliva samples. For example, reduction of steroid biosynthesis may be determined by measuring a steroid intermediate or end product by liquid chromatography/mass spectrometry (LC/MS) or gas chromatography/mass spectrometry (GC/MS).

Administration of a CYP11B1 inhibitor may increase the level of glucocorticoid precursor 11-deoxycortisol in a subject due to it being immediately proximal to the blockage of 11-beta hydroxylase activity. Excess 11-deoxycortisol levels may exhibit mineralocorticoid activity.

The use of a CYP11B1 inhibitor with selective CYP11B1 activity leaves the mineralocorticoid pathway largely intact, which may result in marked increases in aldosterone. The use of a combination of CYP11B1 inhibitor and CYP11B2 inhibitor, either as separate agents or as a dual CYP11B1/CYP11B2 inhibitor agent, further blocks the 11-β-hydroxylase activity of CYP11B2, blocking the conversion of 11-deoxycorticosterone to corticosterone in the aldosterone synthesis pathway. While minimal or no aldosterone is produced using the double blockade of CYP11B1 and CYP11B2, an increased level of 11-deoxycorticosterone is observed. 11-deoxycorticosterone can cause effects of mineralocorticoid excess if it reaches significant levels.

Shunting to the androgen synthesis pathway may increase the production of testosterone, dihydrotestosterone, androstenedione, dehydroepiandrosterone A (DHEA), DHEA-sulfate (DHEA-S), or any combination thereof. Accordingly, in certain embodiments, the androgen or precursor thereof is testosterone, androstenedione, dehydroepiandrosterone A (DHEA), DHEA-S, or any combination thereof.

Shunting to the androgen synthesis pathway may cause various symptoms of androgen excess, for example, acne, hirsutism, virilization, menstrual irregularity, infertility due to anovulation, enlarged clitoris, or male infertility.

In certain embodiments where the method does not employ the inhibition of 11-beta hydroxylase activity of CYP11B2, shunting to the mineralocorticoid synthesis pathway may increase the production of aldosterone, corticosterone, 11-deoxycorticosterone, or any combination thereof. Accordingly, in certain embodiments, the mineralocorticoid or precursor thereof is aldosterone, corticosterone, 11-deoxycorticosterone, or any combination thereof.

Excess mineralocorticoids, such as aldosterone, 11-deoxycorticosterone, or glucocorticoid precursor 11-deoxycortisol with mineralocorticoid activity, may cause hypertension, edema, and hypokalemia.

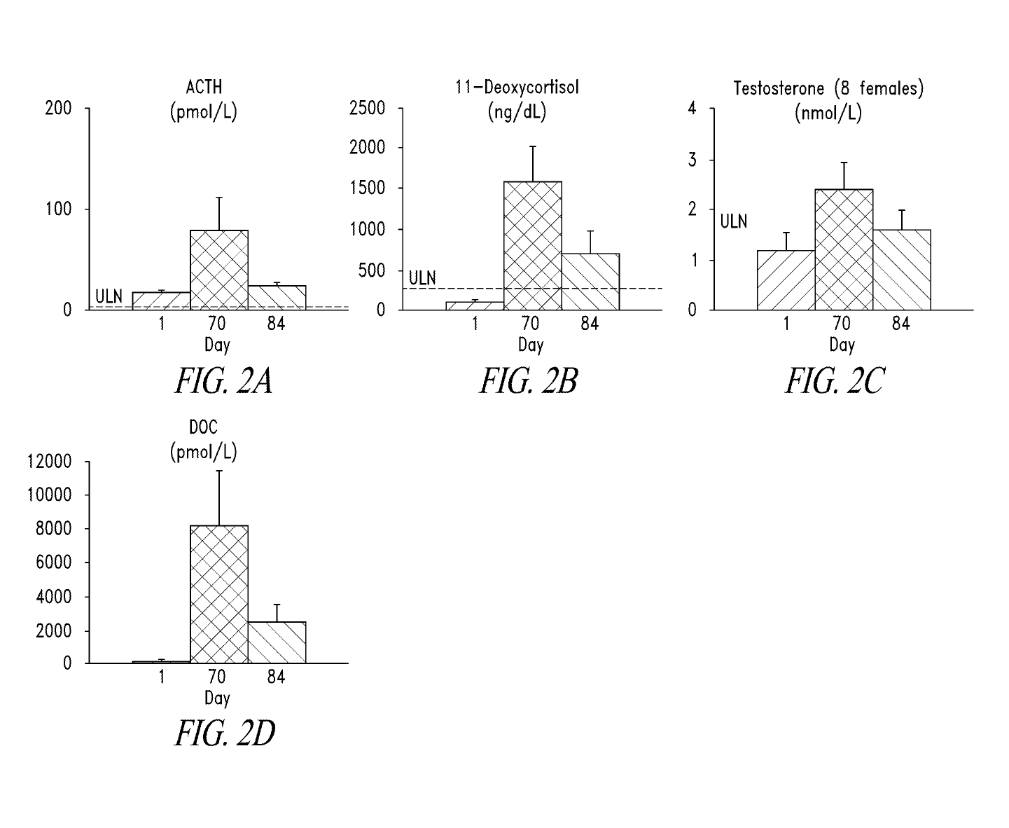

FIGS. 2A-D illustrate effects on the androgen synthesis pathway and mineralocorticoid synthesis pathway in human adult patients with Cushing's disease who are treated with CYP11B1 inhibitor osilodrostat (LCI699). Patients (n=12) were initially administered 4 mg/day (b.i.d.) with dose escalation every 2 weeks until urinary free cortisol (UFC) normalized or the total maximal daily dose was reached (100 mg). Dose was maintained until day 70 and followed by a 2 week washout period until day 84. Hormone levels were measured on days 1, 70, and 84. Day 1 hormone levels represent “before LCI699 treatment.” Day 70 hormone levels represent “during LCI699 treatment.” Day 84 represents “after LCI699 treatment.” During treatment with LCI699, inhibition of cortisol production and resulting decrease in serum cortisol caused increased secretion of ACTH (FIG. 2A). FIG. 2B shows build-up of steroid precursor 11-deoxycortisol before the 11-beta hydroxylase block during treatment with LCI699. FIGS. 2C and 2D show shunting of steroid precursors to androgen pathway and build-up of mineralocorticoid precursor during treatment with LCI699 as demonstrated by increased testosterone levels (in females) and 11-deoxycorticosterone levels, respectively.

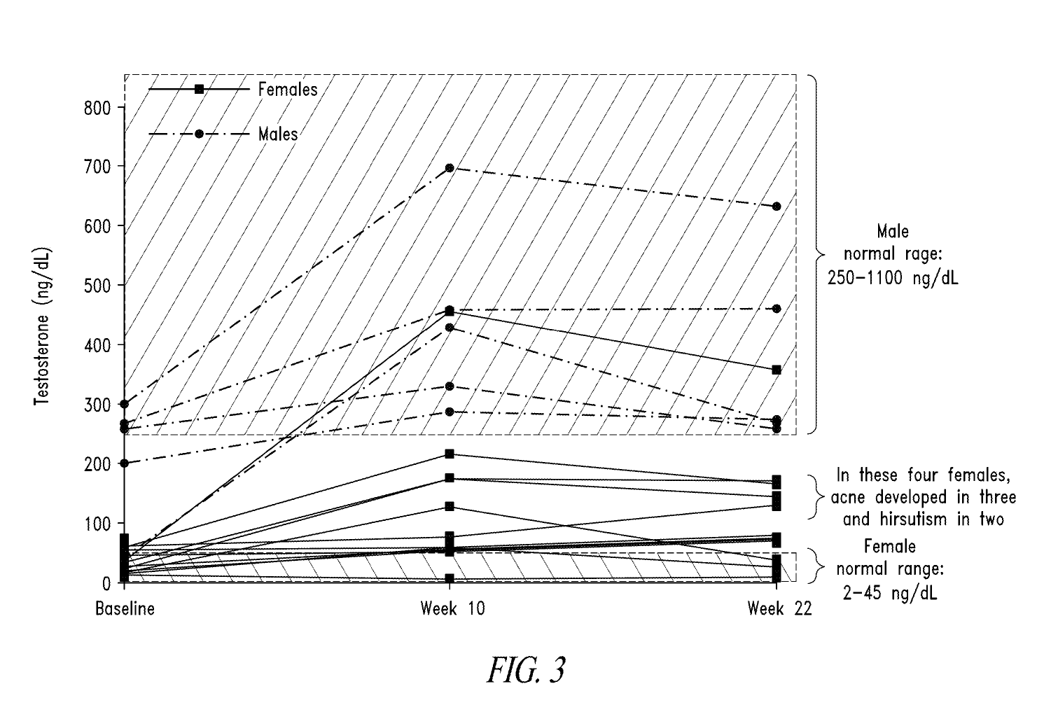

FIG. 3 further illustrates effects of CYP11B1 inhibitor osilodrostat (LCI699) on individual testosterone levels in human, adult patients (5 males, 12 females) with Cushing's disease who completed a 22-week dose-escalation treatment course (Fleseriu et al. 2016, Pituitary 19:138-149). Osilodrostat was initiated at 4 mg/day (b.i.d.) (10 mg/day if UFC>3×ULN) with dose escalated every 2 weeks to 10, 20, 40, and 60 mg/day until UFC≦ULN. 9 of the 12 women who completed the 22 week treatment course exhibited above normal testosterone levels during the treatment course (normal range 2-45 ng/dL) (FIG. 3). Four females with elevated testosterone developed adverse symptoms of androgen excess, with three developing acne and two developing hirsutism.

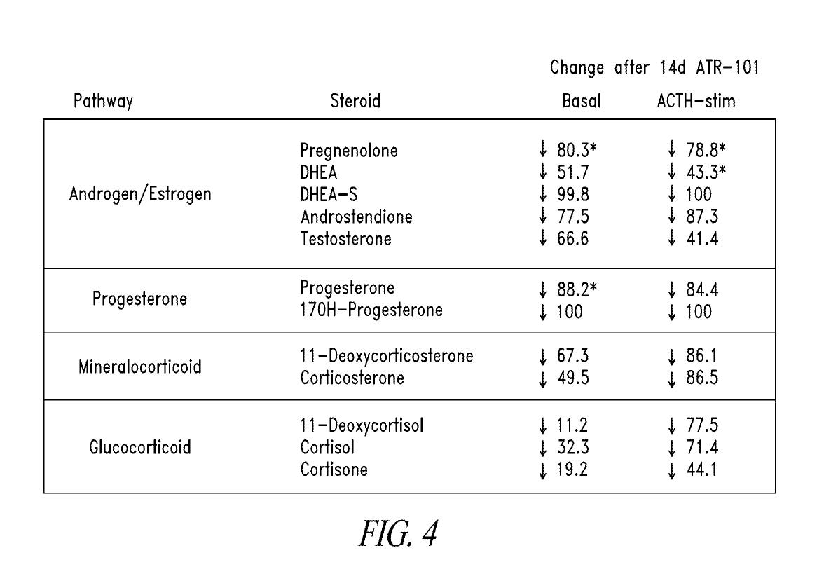

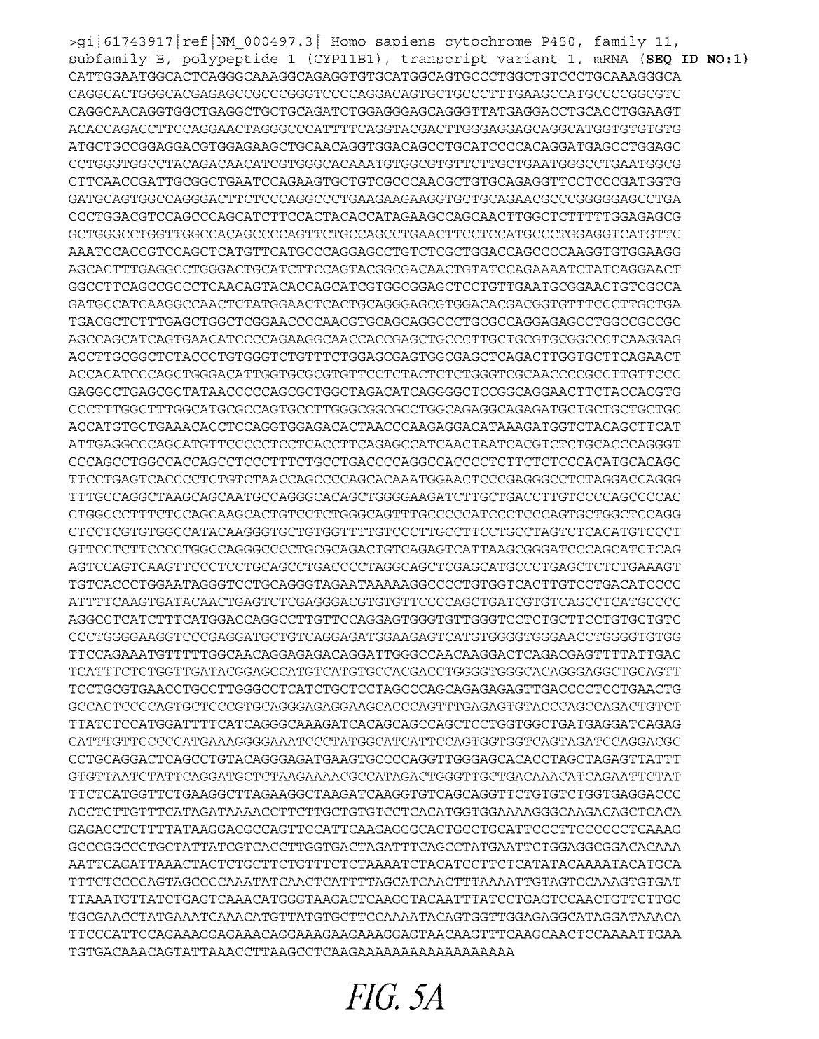

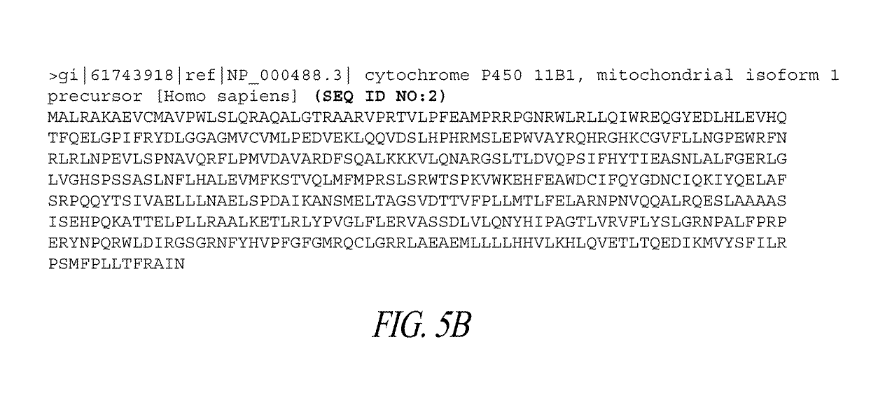

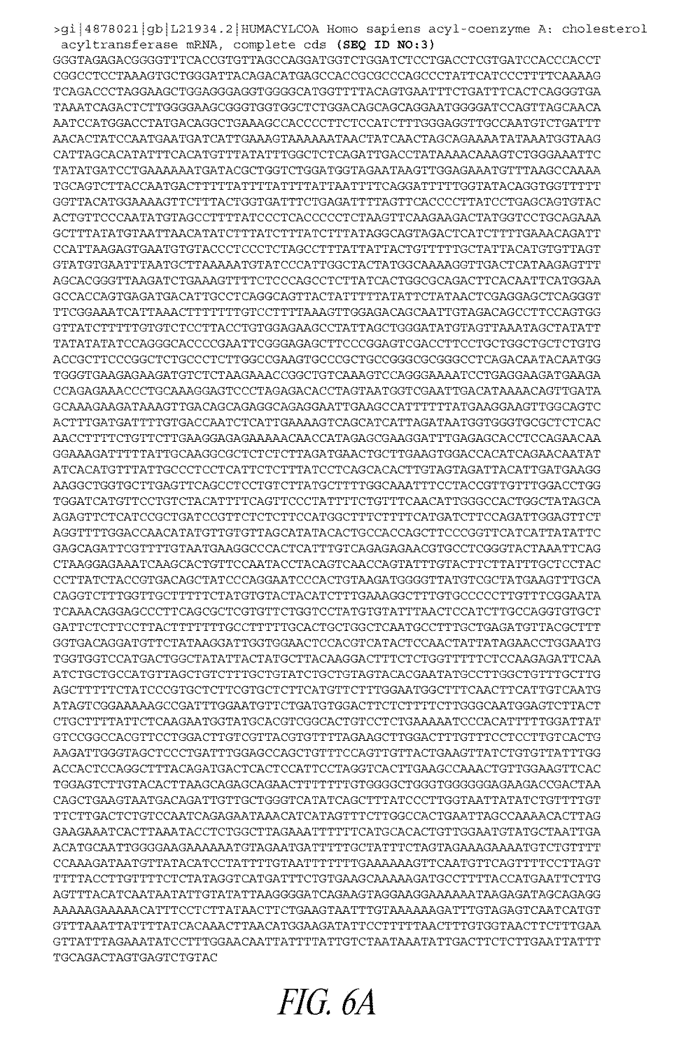

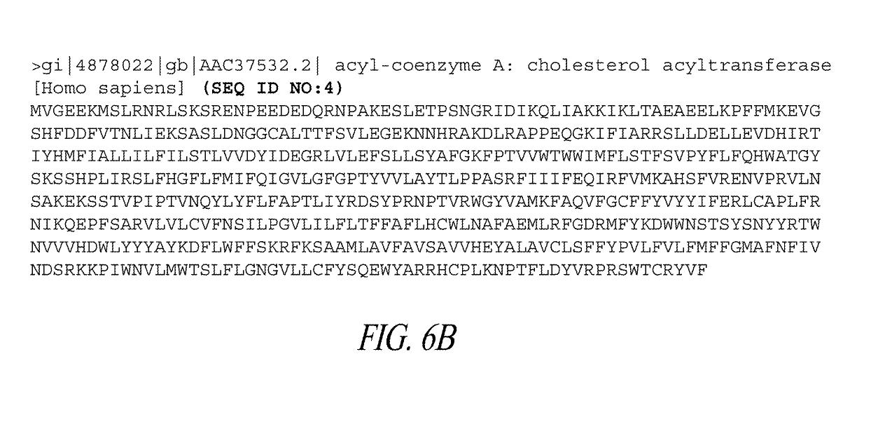

FIG. 4 illustrates the broad inhibitory effects of an ACAT1 inhibitor according to the present disclosure “ATR-101” on synthesis of steroids and steroid precursors produced in the adrenal cortex. Dogs were administered daily doses of 3 mg/kg of ATR-101 for 7 days by oral gavage, and then 30 mg/kg for 7 days by oral gavage. Blood was collected on day 14 to measure basal steroid and steroid precursor serum levels (pre-ACTH stimulation) and steroid/steroid precursor levels post-ACTH stimulation by LC-MS/MS. For ACTH stimulation, 5 μg/kg (not to exceed 250 μg) of CORTROSYN™ (also known as cosyntropin or synthetic ACTH) was administered via bolus i.v. administration, and blood samples were collected 1 hour post-CORTROSYN™ administration. Changes in steroid and steroid precursor levels are shown as % reduction as compared to maximum level as measured on Day 0, except as noted (see, FIG. 2; *Day 1 data used for maximum steroid level; **Day 3 data used for maximum steroid level). Indeed, the very steroid precursor (11-deoxycortisol), androgen (testosterone), and mineralocorticoid (11-deoxycorticosterone) that were shown to be elevated by treatment with a CYP11B1 inhibitor (FIGS. 2A-D, FIG. 3) were decreased in animals treated with ATR-101 (FIG. 4).

In certain embodiments, the administration of the combination of a CYP11B1 inhibitor and ACAT1 inhibitor N-(2,6-bis(1-methylethyl)phenyl)-N′-((1-(4-(dimethylamino)phenyl)cyclopentyl)-methyl)urea or a salt thereof decreases an adverse effect associated with administration of CYP11B1 inhibitor. In certain embodiments, the adverse effect is acne, hirsutism, virilization, menstrual irregularity, infertility due to anovulation, male infertility, enlarged clitoris, hypertension, edema, hypokalemia, gastrointestinal upset, or any combination thereof.

Cholesterol, which has a 17-carbon steroid nucleus, is the precursor of steroid biosynthesis and is converted into steroid hormone intermediates and end products by cytochrome P450 enzymes in the mitochondria and endoplasmic reticulum. Cholesterol may be derived from multiple sources, including de novo synthesis from acetate; absorption as LDLs or HDLs; or lipid droplets containing cholesterol acetate (a cholesterol ester) within adrenocortical cells, which serve as a cholesterol reservoir for steroid biosynthesis. Enzymatic conversion of cholesterol to pregnenalone by CYP11A1 is the rate limiting step for steroid biosynthesis. After synthesis of pregnenalone, synthesis of progestagens, glucocorticoids, mineralocorticoids, androgens, and estrogens may also occur in adrenocortical cells.

Administration of ACAT1 inhibitor N-(2,6-bis(1-methylethyl)phenyl)-N′-((1-(4-(dimethylamino)phenyl)cyclopentyl)-methyl)urea or a salt thereof limits the cholesterol pool (cholesterol ester) that feeds production of androgens, mineralocorticoids, and their precursors. Cholesterol ester levels may be measured by determining total cholesterol and free cholesterol levels as described in Carr et al. (Clin. Biochem. 26:39-42, 1993; hereby incorporated by reference in its entirety). Briefly, lipid extracts are prepared from adrenal glands of treated subjects, and enzymatic assays are used to determine total cholesterol and free cholesterol. Cholesteryl ester is determined by subtracting free cholesterol from total cholesterol. In certain embodiments, administration of the combination of CYP11B1 inhibitor and ACAT1 inhibitor N-(2,6-bis(1-methylethyl)-phenyl)-N′-((1-(4-(dimethylamino)phenyl)cyclopentyl)-methyl)urea or a salt thereof reduces cholesterol ester levels in adrenocortical cells as compared to adrenocortical cells treated with CYP11B1 inhibitor alone.

In certain embodiments, administration of the combination of a CYP11B1 inhibitor and ACAT1 inhibitor N-(2,6-bis(1-methylethyl)phenyl)-N′-((1-(4-(dimethylamino)phenyl)cyclopentyl)-methyl)urea or a salt thereof increases reduction of cortisol biosynthesis as compared to administration of the CYP11B1 inhibitor alone. Methods of measuring reduction of cortisol biosynthesis are known in the art and include liquid chromatography/mass spectrometry.

The agents described herein are administered by any suitable means, either systemically or locally, including via parenteral, subcutaneous, intrapulmonary, intramuscular, oral, and intranasal. Parenteral routes include intravenous, intraarterial, epidural, and intrathecal administration. In various aspects, an agent is administered by pulse infusion. Other administration methods are contemplated, including topical, particularly transdermal, transmucosal, rectal, oral or local administration.

Another aspect of the disclosure provides a pharmaceutical composition comprising a therapeutically effective amount of a combination of a CYP11B1 inhibitor and ACAT1 inhibitor N-(2,6-bis(1-methylethyl)phenyl)-N′-((1-(4-(dimethylamino)-phenyl)cyclopentyl)-methyl)urea or a salt thereof for treating a disorder associated with excess cortisol production. In certain embodiments, the CYP11B1 inhibitor is osilodrostat. In certain other embodiments, the CYP11B1 inhibitor is metyrapone. In certain embodiments, the CYP11B1 inhibitor does not include an adrenolytic agent, e.g., mitotane. In certain embodiments, the disorder associated with excess cortisol production is Cushing's syndrome. In certain embodiments, the CYP11B1 inhibitor and ACAT1 inhibitor are in the same formulation. In other embodiments, the CYP11B1 inhibitor and ACAT1 inhibitor are in separate formulations.

One or more other pharmaceutically acceptable components as described in Remington's Pharmaceutical Sciences 16th edition, Osol, A. Ed. (1980) is included in the formulation provided that they do not adversely affect the desired characteristics of the formulation. Examples of formulations for a pharmaceutical composition include, without limitation, solutions, suspensions, powders, granules, tablets, capsules, pills, lozenges, chews, creams, ointments, gels, liposome preparations, nanoparticulate preparations, injectable preparations, enemas, suppositories, inhalable powders, sprayable liquids, aerosols, patches, depots and implants. In various aspects, a pharmaceutical composition formulation is in the form of a tablet or a capsule. Tablets are, in various aspects, uncoated or comprise a core that is coated, for example with a nonfunctional film or a release-modifying or enteric coating. In various aspects, capsules have hard or soft shells comprising, for example, gelatin and/or HPMC, optionally together with one or more plasticizers. Lyophilized formulations or aqueous solutions are contemplated. Sustained release formulations are also provided.

Various components of a pharmaceutical composition provided depend on the chosen route of administration and desired delivery method.

Suitable carriers include any material which, when combined with the compound, retains the activity and is nonreactive with the subject's immune system. Examples include, but are not limited to, any of a number of standard pharmaceutical carriers. A variety of aqueous carriers are contemplated and include, without limitation, water, buffered water, physiological saline, 0.4% saline, and 0.3% glycine.

In various aspects, a pharmaceutical composition formulation includes a protein for enhanced stability, such as and without limitation, albumin, lipoprotein, and globulin.

In various aspects, a pharmaceutical composition formulation includes a diluent, either individually or in combination, such as, and without limitation, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like.

Diluents, if present, typically constitute in total about 5% to about 99%, about 10% to about 85%, or about 20% to about 80%, by weight of the composition. The diluent or diluents selected exhibit suitable flow properties and, where tablets are desired, compressibility.

In various aspects, a pharmaceutical composition formulation includes binding agents or adhesives which are useful excipients, particularly where the composition is in the form of a tablet. Such binding agents and adhesives should impart sufficient cohesion to the blend being formulated in a tablet to allow for normal processing operations such as sizing, lubrication, compression and packaging, but still allow the tablet to disintegrate and the compound to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, constitute in various aspects, in total about 0.5% to about 25%, for example about 0.75% to about 15%, or about 1% to about 10%, by weight of the composition.

In various aspects, an aqueous pharmaceutical composition formulation of an agent includes a buffer. Examples of buffers include acetate (e.g., sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation. In various aspects, an aqueous pharmaceutical composition formulation of the agent is prepared in a pH-buffered solution, for example, at pH ranging from about 4.5 to about 8.0, or from about 4.8 to about 6.5, or from about 4.8 to about 5.5, or alternatively about 5.0.

In various aspects, a pharmaceutical composition formulation includes a disintegrant.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

In various aspects, a pharmaceutical composition formulation includes a wetting agent. Wetting agents, if present, are normally selected to maintain the compound in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

In various aspects, a pharmaceutical composition formulation includes a lubricant. Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

In various aspects, a pharmaceutical composition formulation includes an anti-adherent. Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

In various aspects, a pharmaceutical composition formulation includes a glidant. Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

In various aspects, a pharmaceutical composition formulation includes a tonicity agent. A tonicity agent may be included in the formulation for stabilization. Exemplary tonicity agents include polyols, such as mannitol, sucrose or trehalose. Preferably, the aqueous formulation is isotonic, although hypertonic or hypotonic solutions are contemplated. Exemplary concentrations of the polyol in the formulation may range from about 1% to about 15% w/v.

In various aspects, a pharmaceutical composition formulation includes a surfactant. A surfactant may also be added to reduce aggregation of the compound and/or to minimize the formation of particulates in the formulation and/or to reduce adsorption. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.01% w/v.

In various aspects, a pharmaceutical composition formulation is essentially free of one or more preservatives, such as benzyl alcohol, phenol, m-cresol, chlorobutanol and benzethonium. In other aspects, a preservative is included in the formulation, e.g., at concentrations ranging from about 0.1% to about 2%, or alternatively from about 0.5% to about 1%.

Sustained-release pharmaceutical composition formulations are also provided. Suitable examples of sustained-release preparations include semipermeable matrices of solid hydrophobic polymers containing the antibody, which matrices are in the form of shaped articles, including without limitation films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The active ingredients may also be entrapped in a microcapsule prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Also provided are pharmaceutical compositions in a lyophilized formulation. The resulting “lyophilized cake” is reconstituted prior to use. Reconstitution of the lyophilized cake adds a volume of aqueous solution, typically equivalent to the volume removed during lyophilization.

The amount of each agent to be administered, and other administration parameters such as frequency and duration of therapy, depend on the agent or prodrug intended for use, and on other factors such as the route of administration, dose intervals, excretion rate, formulation of the agent, the recipient, age, body weight, sex, diet, medical history, and general state (e.g., health) of the subject being treated of the recipient, the severity of the disease, and/or the size, malignancy and invasiveness of a tumor to be treated. The agent is thus administered at a dosage sufficient to achieve a desired therapeutic or prophylactic effect and is determined on a case-by-case basis.

In some embodiments, ACAT1 inhibitor N-(2,6-bis(1-methylethyl)phenyl)-N′-((1-(4-(dimethylamino)phenyl)cyclopentyl)-methyl)urea or a salt thereof is administered at a dosage of about 1.0 μg/kg to about 100 mg/kg, about 0.01 mg/kg to about 100 mg/kg, about 0.01 mg/kg to about 50 mg/kg, about 0.01 mg/kg to about 30 mg/kg, about 0.01 mg/kg to about 10 mg/kg, about 0.01 mg/kg to about 5 mg/kg, about 0.05 mg/kg to about 10 mg/kg, about 0.1 mg/kg to about 200 mg/kg, about 0.1 mg/kg to about 50 mg/kg, about 0.1 mg/kg to about 10 mg/kg, about 0.5 mg/kg to about 25 mg/kg, about 1 mg/kg to about 10 mg/kg, or about 2 mg/kg to about 10 mg/kg.

In some embodiments, a daily dose of at least 0.05 or 1 mg or greater of osilodrostat (LCI699), such as from 0.01 mg to 1000 mg, from 0.01 mg to 500 mg, from 0.01 to 50 mg, from 0.01 mg to 5 mg, from 0.01 to 2 mg or from 0.1 mg to 2 mg of osilodrostat; such as in unit dosage of at least 0.05 or 1 mg or of from 4 mg to 100 mg, for example of from 2 mg to 50 mg, of osilodrostat is administered for a subject of about 50-70 kg. For example, the unit dosage can contain 1-1000 mg of active ingredient for a subject of about 50-70 kg, about 1-500 mg, about 1-50 mg, about 0.5-5 mg, 0.1-1 mg or about 0.05-0.5 mg of active ingredient. In another example, the dosage of osilodrostat that is administered to a subject is from about 2 mg to about 30 mg BID.

In some embodiments, metyrapone is administered at a daily dose from about 0.25 g to about 6 g, or from about 0.5 g to about 5 g, or from about 1 g to about 4.5 g.

Administration is contemplated in a regimen that is daily (once, twice or more per day), alternating days, every third day, or 2, 3, 4, 5, or 6 times per week, weekly, twice a month, monthly or more or less frequently, as necessary, depending on the response or condition and the recipient tolerance of the therapy. Administration of the combination of agents of this disclosure may be as a single dose, or administration may occur several times wherein a plurality of doses is given to a subject in need thereof. The dosage can be increased or decreased over time, as required by an individual patient. In certain instances, a patient initially is given a low dose, which is then increased to an efficacious dosage tolerable to the patient. Determination of an effective amount is well within the capability of those skilled in the art. Maintenance dosages over a longer period of time, such as 4, 5, 6, 7, 8, 10 or 12 weeks or longer are contemplated, and dosages may be adjusted as necessary. The progress of the therapy is monitored by conventional techniques and assays, and is within the skill in the art.

When referring to a combination, a therapeutically effective dose refers to combined amounts of the active ingredients that result in the therapeutic effect, whether administered sequentially or simultaneously (in the same formulation or concurrently in separate formulations). When separate dosage formulations are used, a CYP11B1 inhibitor and ACAT1 inhibitor N-(2,6-bis(1-methylethyl)phenyl)-N′-((1-(4-(dimethylamino)phenyl)-cyclopentyl)-methyl)urea or a salt thereof can be administered at essentially the same time, i.e., concurrently, or at separately staggered times, i.e., sequentially; combination therapy is understood to include all these regimens. For example, a CYP11B1 inhibitor and ACAT1 inhibitor N-(2,6-bis(1-methylethyl)phenyl)-N′-((1-(4-(dimethylamino)phenyl)cyclopentyl)-methyl)urea or a salt thereof can be administered to the patient together in a single oral dosage composition, such as a tablet or capsule, or each agent may be administered in separate oral dosage formulations. In certain embodiments, the CYP11B1 inhibitor and ACAT1 inhibitor N-(2,6-bis(1-methylethyl)phenyl)-N′-((1-(4-(dimethylamino)phenyl)-cyclopentyl)-methyl)urea or a salt thereof are administered simultaneously. In other embodiments, the CYP11B1 inhibitor and ACAT1 inhibitor N-(2,6-bis(1-methylethyl)-phenyl)-N′-((1-(4-(dimethylamino)phenyl)cyclopentyl)-methyl)urea or a salt thereof are administered sequentially.

Kits with unit doses of the combination of agents described herein, usually in oral or injectable doses, are provided for use in treating a disorder associated with excess cortisol production in a subject in need thereof. Unit doses of each agent may be provided in separate formulations or in the same formulation. Such kits may include a container containing the unit dose, an informational package insert describing the use and attendant benefits of the drugs in treating the disorder associated with excess cortisol production, and optionally an appliance or device for delivery of the composition.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating a disorder associated with excess cortisol production in a subject in need thereof, comprising administering to a subject a therapeutically effective amount of a combination of: (a) a CYP11B1 inhibitor; and (b) an ACAT1 inhibitor, wherein the ACAT1 inhibitor is N-(2,6-bis(1-methylethyl)phenyl)-N′-((1-(4-(dimethylamino)phenyl)-cyclopentyl)-methyl)urea or a salt thereof.

2. The method of claim 1, further comprising administering a CYP11B2 inhibitor.

3. The method of claim 2, wherein the CYP11B1 inhibitor and CYP11B2 are a dual CYP11B1/CYP11B2 inhibitor.

4. The method of claim 1, wherein the CYP11B1 inhibitor is osilodrostat.

5. The method of claim 1, wherein the CYP11B1 inhibitor is metyrapone.

6. The method of claim 1, wherein the CYP11B1 inhibitor is not mitotane.

7. The method of claim 1, wherein the disorder associated with excess cortisol production is Cushing's syndrome.

8. The method of claim 1, wherein administration of the combination of CYP11B1 inhibitor and ACAT1 inhibitor decreases the production or activity of at least one glucocorticoid precursor, androgen or precursor thereof, mineralocorticoid or a precursor thereof, or any combination thereof as compared to administration of the CYP11B1 inhibitor alone.

9. The method according to claim 8, wherein the androgen or precursor thereof is testosterone, androstenedione, DHEA, DHEA-S, or a combination thereof.

10. The method according to claim 8, wherein the mineralocorticoid or precursor thereof is corticosterone, 11-deoxycorticosterone, aldosterone, or a combination thereof.

11. The method of claim 8, wherein the glucocorticoid precursor is 11-deoxycortisol.

12. The method of claim 1, wherein the administration of the combination of CYP11B1 inhibitor and ACAT1 inhibitor decreases an adverse effect associated with administration of CYP11B1 inhibitor alone.

13. The method according to claim 12, wherein the adverse effect is acne, hirsutism, virilization, menstrual irregularity, infertility due to anovulation, male infertility, enlarged clitoris, hypertension, edema, hypokalemia, or any combination thereof.

14. The method of claim 1, wherein the administration of the combination of CYP11B1 inhibitor and ACAT1 inhibitor reduces cholesterol ester levels in adrenocortical cells as compared to adrenocortical cells treated with CYP11B1 inhibitor alone.

15. The method of claim 1, wherein the administration of the combination of CYP11B1 inhibitor and ACAT1 inhibitor increases reduction of cortisol biosynthesis as compared to administration of the CYP11B1 inhibitor alone.

16. The method of claim 1, wherein the CYP11B1 inhibitor and ACAT1 inhibitor are administered simultaneously or sequentially.

17. The method of claim 16, wherein the CYP11B1 inhibitor and ACAT1 inhibitor are administered in separate formulations.

18. The method of claim 16, wherein the CYP11B1 inhibitor and ACAT inhibitor are administered simultaneously in the same formulation.


SEQUENCE LISTING (6 sequences)

SEQ ID NO: 1 - DNA, 3551 bp, Homo sapiens
cattggaatggcactcagggcaaaggcagaggtgtgcatggcagtgccctggctgtccctgcaaagggcacaggcactgggcacgagagccgcccgggtccccaggacagtgctgccctttgaagccatgccccggcgtccaggcaacaggtggctgaggctgctgcagatctggagggagcagggttatgaggacctgcacctggaagtacaccagaccttccaggaactagggcccattttcaggtacgacttgggaggagcaggcatggtgtgtgtgatgctgccggaggacgtggagaagctgcaacaggtggacagcctgcatccccacaggatgagcctggagccctgggtggcctacagacaacatcgtgggcacaaatgtggcgtgttcttgctgaatgggcctgaatggcgcttcaaccgattgcggctgaatccagaagtgctgtcgcccaacgctgtgcagaggttcctcccgatggtggatgcagtggccagggacttctcccaggccctgaagaagaaggtgctgcagaacgcccgggggagcctgaccctggacgtccagcccagcatcttccactacaccatagaagccagcaacttggctctttttggagagcggctgggcctggttggccacagccccagttctgccagcctgaacttcctccatgccctggaggtcatgttcaaatccaccgtccagctcatgttcatgcccaggagcctgtctcgctggaccagccccaaggtgtggaaggagcactttgaggcctgggactgcatcttccagtacggcgacaactgtatccagaaaatctatcaggaactggccttcagccgccctcaacagtacaccagcatcgtggcggagctcctgttgaatgcggaactgtcgccagatgccatcaaggccaactctatggaactcactgcagggagcgtggacacgacggtgtttcccttgctgatgacgctctttgagctggctcggaaccccaacgtgcagcaggccctgcgccaggagagcctggccgccgcagccagcatcagtgaacatccccagaaggcaaccaccgagctgcccttgctgcgtgcggccctcaaggagaccttgcggctctaccctgtgggtctgtttctggagcgagtggcgagctcagacttggtgcttcagaactaccacatcccagctgggacattggtgcgcgtgttcctctactctctgggtcgcaaccccgccttgttcccgaggcctgagcgctataacccccagcgctggctagacatcaggggctccggcaggaacttctaccacgtgccctttggctttggcatgcgccagtgccttgggcggcgcctggcagaggcagagatgctgctgctgctgcaccatgtgctgaaacacctccaggtggagacactaacccaagaggacataaagatggtctacagcttcatattgaggcccagcatgttccccctcctcaccttcagagccatcaactaatcacgtctctgcacccagggtcccagcctggccaccagcctccctttctgcctgaccccaggccacccctcttctctcccacatgcacagcttcctgagtcacccctctgtctaaccagccccagcacaaatggaactcccgagggcctctaggaccagggtttgccaggctaagcagcaatgccagggcacagctggggaagatcttgctgaccttgtccccagccccacctggccctttctccagcaagcactgtcctctgggcagtttgcccccatccctcccagtgctggctccaggctcctcgtgtggccatacaagggtgctgtggttttgtcccttgccttcctgcctagtctcacatgtccctgttcctcttcccctggccagggcccctgcgcagactgtcagagtcattaagcgggatcccagcatctcagagtccagtcaagttccctcctgcagcctgacccctaggcagctcgagcatgccctgagctctctgaaagttgtcaccctggaatagggtcctgcagggtagaataaaaaggcccctgtggtcacttgtcctgacatccccattttcaagtgatacaactgagtctcgagggacgtgtgttccccagctgatcgtgtcagcctcatgccccaggcctcatctttcatggaccaggccttgttccaggagtgggtgttgggtcctctgcttcctgtgctgtcccctggggaaggtcccgaggatgctgtcaggagatggaagagtcatgtggggtgggaacctggggtgtggttccagaaatgtttttggcaacaggagagacaggattgggccaacaaggactcagacgagttttattgactcatttctctggttgatacggagccatgtcatgtgccacgacctggggtgggcacagggaggctgcagtttcctgcgtgaacctgccttgggcctcatctgctcctagcccagcagagagagttgacccctcctgaactggccactccccagtgctcccgtgcagggagaggaagcacccagtttgagagtgtacccagccagactgtctttatctccatggattttcatcagggcaaagatcacagcagccagctcctggtggctgatgaggatcagagcatttgttcccccatgaaaggggaaatccctatggcatcattccagtggtggtcagtagatccaggacgccctgcaggactcagcctgtacagggagatgaagtgccccaggttgggagcacacctagctagagttatttgtgttaatctattcaggatgctctaagaaaacgccatagactgggttgctgacaaacatcagaattctatttctcatggttctgaaggcttagaaggctaagatcaaggtgtcagcaggttctgtgtctggtgaggacccacctcttgtttcatagataaaaccttcttgctgtgtcctcacatggtggaaaagggcaagacagctcacagagacctcttttataaggacgccagttccattcaagagggcactgcctgcattcccttcccccctcaaaggcccggccctgctattatcgtcaccttggtgactagatttcagcctatgaattctggaggcggacacaaaaattcagattaaactactctgcttctgtttctctaaaatctacatccttctcatatacaaaatacatgcatttctccccagtagccccaaatatcaactcattttagcatcaactttaaaattgtagtccaaagtgtgatttaaatgttatctgagtcaaacatgggtaagactcaaggtacaatttatcctgagtccaactgttcttgctgcgaacctatgaaatcaaacatgttatgtgcttccaaaatacagtggttggagaggcataggataaacattcccattccagaaaggagaaacaggaaagaagaaaggagtaacaagtttcaagcaactccaaaattgaatgtgacaaacagtattaaaccttaagcctcaagaaaaaaaaaaaaaaaaaa

SEQ ID NO: 2 - protein, 503 aa, Homo sapiens
MALRAKAEVCMAVPWLSLQRAQALGTRAARVPRTVLPFEAMPRRPGNRWLRLLQIWREQGYEDLHLEVHQTFQELGPIFRYDLGGAGMVCVMLPEDVEKLQQVDSLHPHRMSLEPWVAYRQHRGHKCGVFLLNGPEWRFNRLRLNPEVLSPNAVQRFLPMVDAVARDFSQALKKKVLQNARGSLTLDVQPSIFHYTIEASNLALFGERLGLVGHSPSSASLNFLHALEVMFKSTVQLMFMPRSLSRWTSPKVWKEHFEAWDCIFQYGDNCIQKIYQELAFSRPQQYTSIVAELLLNAELSPDAIKANSMELTAGSVDTTVFPLLMTLFELARNPNVQQALRQESLAAAASISEHPQKATTELPLLRAALKETLRLYPVGLFLERVASSDLVLQNYHIPAGTLVRVFLYSLGRNPALFPRPERYNPQRWLDIRGSGRNFYHVPFGFGMRQCLGRRLAEAEMLLLLHHVLKHLQVETLTQEDIKMVYSFILRPSMFPLLTFRAIN

SEQ ID NO: 3 - DNA, 4011 bp, Homo sapiens
gggtagagacggggtttcaccgtgttagccaggatggtctggatctcctgacctcgtgatccacccacctcggcctcctaaagtgctgggattacagacatgagccaccgcgcccagccctattcatcccttttcaaaagtcagaccctaggaagctggagggaggtggggcatggttttacagtgaatttctgatttcactcagggtgataaatcagactcttggggaagcgggtggtggctctggacagcagcaggaatggggatccagttagcaacaaatccatggacctatgacaggctgaaagccaccccttctccatctttgggaggttgccaatgtctgatttaacactatccaatgaatgatcattgaaagtaaaaaataactatcaactagcagaaaatataaatggtaagcattagcacatatttcacatgtttatatttggctctcagattgacctataaaacaaagtctgggaaattctatatgatcctgaaaaaatgatacgctggtctggatggtagaataagttggagaaatgtttaagccaaaatgcagtcttaccaatgactttttattttattttattaattttcaggatttttggtatacaggtggtttttggttacatggaaaagttctttactggtgatttctgagattttagttcaccccttatcctgagcagtgtacactgttcccaatatgtagccttttatccctcaccccctctaagttcaagaagactatggtcctgcagaaagctttatatgtaattaacatatctttatctttatctttataggcagtagactcatcttttgaaacagattccattaagagtgaatgtgtaccctccctctagcctttattattactgtttttgctattacatgtgttagtgtatgtgaatttaatgcttaaaaatgtatcccattggctactatggcaaaaggttgactcataagagtttagcacgggttaagatctgaaagttttctcccagcctcttatcactggcgcagacttcacaattcatggaagccaccagtgagatgacattgcctcaggcagttactatttttatattctataactcgaggagctcagggtttcggaaatcattaaactttttttgtccttttaaagttggagacagcaattgtagacagccttccagtgggttatctttttgtgtctccttacctgtggagaagcctattagctgggatatgtagttaaatagctatatttatatatatccagggcaccccgaattcgggagagcttcccggagtcgaccttcctgctggctgctctgtgaccgcttcccggctctgccctcttggccgaagtgcccgctgccgggcgcgggcctcagacaatacaatggtgggtgaagagaagatgtctctaagaaaccggctgtcaaagtccagggaaaatcctgaggaagatgaagaccagagaaaccctgcaaaggagtccctagagacacctagtaatggtcgaattgacataaaacagttgatagcaaagaagataaagttgacagcagaggcagaggaattgaagccattttttatgaaggaagttggcagtcactttgatgattttgtgaccaatctcattgaaaagtcagcatcattagataatggtgggtgcgctctcacaaccttttctgttcttgaaggagagaaaaacaaccatagagcgaaggatttgagagcacctccagaacaaggaaagatttttattgcaaggcgctctctcttagatgaactgcttgaagtggaccacatcagaacaatatatcacatgtttattgccctcctcattctctttatcctcagcacacttgtagtagattacattgatgaaggaaggctggtgcttgagttcagcctcctgtcttatgcttttggcaaatttcctaccgttgtttggacctggtggatcatgttcctgtctacattttcagttccctattttctgtttcaacattgggccactggctatagcaagagttctcatccgctgatccgttctctcttccatggctttcttttcatgatcttccagattggagttctaggttttggaccaacatatgttgtgttagcatatacactgccaccagcttcccggttcatcattatattcgagcagattcgttttgtaatgaaggcccactcatttgtcagagagaacgtgcctcgggtactaaattcagctaaggagaaatcaagcactgttccaatacctacagtcaaccagtatttgtacttcttatttgctcctacccttatctaccgtgacagctatcccaggaatcccactgtaagatggggttatgtcgctatgaagtttgcacaggtctttggttgctttttctatgtgtactacatctttgaaaggctttgtgcccccttgtttcggaatatcaaacaggagcccttcagcgctcgtgttctggtcctatgtgtatttaactccatcttgccaggtgtgctgattctcttccttactttttttgcctttttgcactgctggctcaatgcctttgctgagatgttacgctttggtgacaggatgttctataaggattggtggaactccacgtcatactccaactattatagaacctggaatgtggtggtccatgactggctatattactatgcttacaaggactttctctggtttttctccaagagattcaaatctgctgccatgttagctgtctttgctgtatctgctgtagtacacgaatatgccttggctgtttgcttgagctttttctatcccgtgctcttcgtgctcttcatgttctttggaatggctttcaacttcattgtcaatgatagtcggaaaaagccgatttggaatgttctgatgtggacttctcttttcttgggcaatggagtcttactctgcttttattctcaagaatggtatgcacgtcggcactgtcctctgaaaaatcccacatttttggattatgtccggccacgttcctggacttgtcgttacgtgttttagaagcttggactttgtttcctccttgtcactgaagattgggtagctccctgatttggagccagctgtttccagttgttactgaagttatctgtgttatttggaccactccaggctttacagatgactcactccattcctaggtcacttgaagccaaactgttggaagttcactggagtcttgtacacttaagcagagcagaactttttttgtggggctgggtggggggagaagaccgactaacagctgaagtaatgacagattgttgctgggtcatatcagctttatcccttggtaattatatctgttttgtttcttgactctgtccaatcagagaataaacatcatagtttcttggccactgaattagccaaaacacttaggaagaaatcacttaaatacctctggcttagaaattttttcatgcacactgttggaatgtatgctaattgaacatgcaattggggaagaaaaaatgtagaatgatttttgctatttctagtagaaagaaaatgtctgttttccaaagataatgttatacatcctattttgtaatttttttgaaaaaagttcaatgttcagttttccttagtttttaccttgttttctctataggtcatgatttctgtgaagcaaaaagatgccttttaccatgaattcttgagtttacatcaataatattgtatattaaggggatcagaagtaggaaggaaaaaataagagatagcagaggaaaaagaaaaacatttcctcttataacttctgaagtaatttgtaaaaaagatttgtagagtcaatcatgtgtttaaattattttatcacaaacttaacatggaagatattcctttttaactttgtggtaacttctttgaagttatttagaaatatcctttggaacaattattttattgtctaataaatattgacttctcttgaattattttgcagactagtgagtctgtac

SEQ ID NO: 4 - protein, 550 aa, Homo sapiens
MVGEEKMSLRNRLSKSRENPEEDEDQRNPAKESLETPSNGRIDIKQLIAKKIKLTAEAEELKPFFMKEVGSHFDDFVTNLIEKSASLDNGGCALTTFSVLEGEKNNHRAKDLRAPPEQGKIFIARRSLLDELLEVDHIRTIYHMFIALLILFILSTLVVDYIDEGRLVLEFSLLSYAFGKFPTVVWTWWIMFLSTFSVPYFLFQHWATGYSKSSHPLIRSLFHGFLFMIFQIGVLGFGPTYVVLAYTLPPASRFIIIFEQIRFVMKAHSFVRENVPRVLNSAKEKSSTVPIPTVNQYLYFLFAPTLIYRDSYPRNPTVRWGYVAMKFAQVFGCFFYVYYIFERLCAPLFRNIKQEPFSARVLVLCVFNSILPGVLILFLTFFAFLHCWLNAFAEMLRFGDRMFYKDWWNSTSYSNYYRTWNVVVHDWLYYYAYKDFLWFFSKRFKSAAMLAVFAVSAVVHEYALAVCLSFFYPVLFVLFMFFGMAFNFIVNDSRKKPIWNVLMWTSLFLGNGVLLCFYSQEWYARRHCPLKNPTFLDYVRPRSWTCRYVF

SEQ ID NO: 5 - DNA, 2936 bp, Homo sapiens
ggaatggcactcagggcaaaggcagaggtgtgcgtggcagcgccctggctgtccctgcaaagggcacgggcactgggcactagagccgctcgggcccctaggacggtgctgccgtttgaagccatgccccagcatccaggcaacaggtggctgaggctgctgcagatctggagggagcagggttatgagcacctgcacctggagatgcaccagaccttccaggagctggggcccattttcaggtacaacttgggaggaccacgcatggtgtgtgtgatgctgccggaggatgtggagaagctgcaacaggtggacagcctgcatccctgcaggatgatcctggagccctgggtggcctacagacaacatcgtgggcacaaatgtggcgtgttcttgttgaatgggcctgaatggcgcttcaaccgattgcggctgaacccagatgtgctgtcgcccaaggccgtgcagaggttcctcccgatggtggatgcagtggccagggacttctcccaggccctgaagaagaaggtgctgcagaacgcccgggggagcctgaccctggacgtccagcccagcatcttccactacaccatagaagccagcaacttagctctttttggagagcggctgggcctggttggccacagccccagttctgccagcctgaacttcctccatgccctggaggtcatgttcaaatccaccgtccagctcatgttcatgcccaggagcctgtctcgctggatcagccccaaggtgtggaaggagcactttgaggcctgggactgcatcttccagtacggtgacaactgtatccagaaaatctaccaggaactggccttcaaccgccctcaacactacacaggcatcgtggcggagctcctgttgaaggcggaactgtcactagaagccatcaaggccaactctatggaactcactgcagggagcgtggacacgacagcgtttcccttgctgatgacgctctttgagctggctcggaaccccgacgtgcagcagatcctgcgccaggagagcctggccgccgcagccagcatcagtgaacatccccagaaggcaaccaccgagctgcccttgctgcgggcggccctcaaggagaccttgcggctctaccctgtgggtctgtttttggagcgagtggtgagctcagacttggtgcttcagaactaccacatcccagctgggacattggtacaggttttcctctactcgctgggtcgcaatgccgccttgttcccgaggcctgagcggtataatccccagcgctggctagacatcaggggctccggcaggaacttccaccacgtgccctttggctttggcatgcgccagtgcctcgggcggcgcctggcagaggcagagatgctgctgctgctgcaccacgtgctgaagcacttcctggtggagacactaactcaagaggacataaagatggtctacagcttcatattgaggcctggcacgtcccccctcctcactttcagagcgattaactagtcttgcatctgcacccagggtcccagcctggccaccagcttccctctgcctgaccccaggccacctgtcttctctcccacgtgcacagcttcctgagtcacccctctgtccagccagctcctgcacaaatggaactccccagggcctccaggactggggcttgccaggcttgtcaaatagcaaggccagggcacagctggagacgatcttgctggcagggcctggccttgtccccagctccacctggccccttctccagcaagcagtgccctctggacagcttgactctactcccagcgctggctccaggctcctcatgaggccatgcaagggtgctgtgattttgtcccttgccttcctgcctagtctcacatgtccctgtccctctcgccctggccagggcctctgtgcagacagtgtcagagtcattaagcgggatcccagcatctcagagtccagtcaagttccctcctgcagcctgaccccaggcagctcgagcatgccctgagctctctgaaagttgtcaccctggaatacgatcctgcagggtagactaaaaaggcccctgtggtcacttatcctgacacattttcaagtgatacaactgagtctcgaggggcgtgtgttccccagctgatcatgtcagcctcatgccccaggcctcgtctttcatggaccaggtcttgttcaagcagcgagtgttgggtcctctgcttcctgagctgtcccctggagaaggtcccgaggatgctgtcaggagatggaagagtcatgtggggtgggaacctggggtgtggttccagaaatgtttttggcaacaggagagacaggattgggccaacaaggactcagatgagttttattgactcattcctctggttgatacggagccatgtcatgtgccacgacctggggtgggcacagggaggctgcagttccctacgtgaacctgccttgggcctcatctgctcctagcccagcagagagagttgacccctcctgaactggccactccccagtgctcctgtgcagggataggaagtgccccagggtgagaacgtgcccagccacatcatctttatctcctgggattttcattagggcaaagatctcagcagccagctcctggtagctgatgaggatcagagcgtttgttcccccatgaaaggggaaatacttaggtaatcattccaggtgtgttcagtagttccaggactcagggactcaggccagtcaccctgtgaccgcaggttgctttcccaccctgggcaatgcagtgcagcatgggaaaggaataagggggcaacaaggtgcacagacctcagagatggcttccttgtttatggggctctcacagatacccatccaactcctctagtcagcactagatcatgggatcctaaaataaaccttggaaaaat

SEQ ID NO: 6 - protein, 503 aa, Homo sapiens
MALRAKAEVCVAAPWLSLQRARALGTRAARAPRTVLPFEAMPQHPGNRWLRLLQIWREQGYEHLHLEMHQTFQELGPIFRYNLGGPRMVCVMLPEDVEKLQQVDSLHPCRMILEPWVAYRQHRGHKCGVFLLNGPEWRFNRLRLNPDVLSPKAVQRFLPMVDAVARDFSQALKKKVLQNARGSLTLDVQPSIFHYTIEASNLALFGERLGLVGHSPSSASLNFLHALEVMFKSTVQLMFMPRSLSRWISPKVWKEHFEAWDCIFQYGDNCIQKIYQELAFNRPQHYTGIVAELLLKAELSLEAIKANSMELTAGSVDTTAFPLLMTLFELARNPDVQQILRQESLAAAASISEHPQKATTELPLLRAALKETLRLYPVGLFLERVVSSDLVLQNYHIPAGTLVQVFLYSLGRNAALFPRPERYNPQRWLDIRGSGRNFHHVPFGFGMRQCLGRRLAEAEMLLLLHHVLKHFLVETLTQEDIKMVYSFILRPGTSPLLTFRAIN